(19) United States
(12) Patent Application Publication
(10) Pub. No.: US 2015/0328073 A1
(43) Pub. Date: Nov. 19, 2015

(54) Hyperbaric Social Establishment or Residence

(71) Applicant: Joseph Gerard Archer, Hernandez, NM (US)

(72) Inventor: Joseph Gerard Archer, Hernandez, NM (US)

(21) Appl. No.: 14/281,503

(22) Filed: May 19, 2014

(51) Int. Cl. A61G 10/02; G06Q 50/16; A61M 16/12; G06Q 90/00

(52) U.S. Cl. CPC A61G 10/026; G06Q 90/00; G06Q 50/16; A61M 16/12

(57) ABSTRACT

A hyperbaric social establishment or residence consists of a social establishment or residence that maintains a hyperbaric atmosphere in which the clientele or residents are immersed. The breathable atmosphere of the establishment is pressurized above standard atmospheric pressure and the ratios of constituent gases are controlled so that the partial pressure of oxygen and the remaining gases are controlled at levels not considered to have any unacceptable long term harmful effects on humans. This establishment or residence is intended to offer to the public a means of obtaining a temporary alcohol-like effect using a pressurized breathable atmosphere instead of alcohol. This temporary inebriating effect is superior to alcohol as it eliminates a hangover, liver and brain damage, and personal and legal complications associated with alcohol or drug use. It is also intended for the comfort of people who suffer from Chronic Obstructive Pulmonary Disease.

### BACKGROUND OF THE INVENTION

1. Field of the Invention

The present invention generally relates to the sale of a commodity and specifically to the commodity of a modified breathable atmospheric within a public social establishment or residence. It is the use of hyperbaric technology to provide an alternative to alcohol consumption and to provide comfort for those suffering from Chronic Obstructive Pulmonary Disease (COPD).

The desire by humans to self-medicate by engaging in self-intoxication is a near universal condition outside of the public element of Muslim countries. The problem is that the intoxicating agents available such as alcohol or additional legal and illegal pharmaceuticals have serious unavoidable collateral hazards. In contrast, the breathing of numerous inert gases at elevated atmospheric pressures provide inebriating effects with no known, and presumably much lower collateral hazard. The principle complication is the added difficulty of providing an atmosphere in a comfortable setting at a sufficiently elevated pressure. The invention presented here is the creation of such an atmosphere specifically to address the need for a superior alternative to alcohol as a method of publically acceptable intoxication.

In the treatment of people suffering from COPD, the partial pressure of oxygen is increased for the patient by providing oxygen at an elevated concentration. This requires wearing a nasal cannula and the use of an oxygen concentrator or oxygen tanks. The use of elevated pressure has not been used to increase the partial pressure of oxygen to a COPD patient.

2. Description of Related Art

The idea of using a hyperbaric atmosphere for the purpose of decompressing a human acclimated to a high pressure atmosphere associated with scuba diving is well established. In addition, the use of non-normal atmospheres such as helium is also well established. However, the use of both pressure and specific gases as a means of providing a non-alcoholic alternative of public entertainment has not been implemented and is not obvious.

### SUMMARY OF THE INVENTION

In accordance with the teachings of the present invention, a hyperbaric social establishment or residence (HSEOR) would consist of an enclosed space designed to accommodate patrons in a comfortable environment and capable of containing a breathable atmosphere at a pressure greater than normal atmospheric pressure. A HSEOR would consist of a main chamber capable of containing pressure. This main chamber may or may not be connected to one of more air locks. An air lock would consist of two doors with a space between them. One door of the air lock would access the outside while the other door would access the main chamber.

Access into the main chamber would be unrestricted. In the absence of an air lock, the pressurizable main chamber would be pressurized after entry. To enter a pressurized chamber would only require passing through an air lock one door at a time with no mandatory wait time between passage through the outside door and passage through the main chamber door. Egress from the main chamber, however, would be restricted. It would require that a person spend some time at a partially elevated pressure to allow dine for the inert gases in the body to he released from the blood stream at a safe rate.

Typically, the pressure in the main chamber might range from 2-6 bar gauge pressure whereas the pressure upon egress during the mandatory wait interval would be approximately 1 bar gauge pressure. The variable gas pressure of the main chamber or multiple chambers would allow for differing levels of desired intoxication.

The principle objective of the invention is to provide a controlled level of a relatively benign and quickly reversible intoxication. An HSEOR would offer a narcosis based form of relaxation and recreation and provide an alternative for people seeking a non-alcoholic or non-drug alternative to an alcohol-like state of mind.

Another objective of the present invention is to provide to the public, a means of becoming intoxicated that has no known longer term detrimental effects on the body such as a hangover, liver damage, or brain damage.

Another objective of the present invention is to provide the public a means of enjoying an evening of intoxication in a public venue that eliminates the need to commute by taxi or if a taxi is not available, to take a risk of being charged with a criminal offense of public intoxication or drunk driving.

Another objective of the present invention is to provide people suffering from COPD means to increase the partial pressure of oxygen to their lungs without having to deal with a nasal cannula and oxygen concentrator or oxygen tank. There are a large number of people who would benefit by being able to deal with their COPD in a comfortable manner.

Another objective of the present invention is to take advantage of financial real estate opportunities appropriate to and only revealed by the unique engineering requirements of a HSEOR

In the ideal manifestation of this invention, a HSEOR would be a cavity carved out of the earth at a sufficient depth that the pressure exerted by the overburden would be approximately equal to the desired atmospheric pressure within the social establishment. This requirement, that the HSEOR be able to contain the extreme force of a pressurized gas distributed over large surface areas, reveals an alternate value of this invention, the commercialization of subterranean real estate. In that a HSEOR is most economically achievable by underground excavation that is at least 50 feet below ground reveals one of its greatest financial advantages; the concept of utilizing readily available and ideally located real estate that heretofore has no commercial value.

The most marketable element of a social establishment is location. Worldwide, real estate near the centers of major cities like Tokyo or New York is either unavailable or unaffordable. The concept of utilizing subterranean real estate has only been used for the construction of subways. Although buildings usually build sub-basements or underground garages, the idea of leasing space deep below a surface structure to construct a separate business or residence has never been considered and is a specific and separate innovation claimed in this application.

The innovation of tying subterranean real estate to a HSEOR lends itself to being able to place any such HSEOR in the very best locations around the world at the very least in real estate costs for little more than the cost of excavating a few hundred feet of rock. Just as importantly, a HSEOR being located at the approximate elevation of a subway also lends itself to easy access by a subway; an additional financial advantage.

In the preferred embodiment, the establishment would be able to obtain permits to replace a portion of the nitrogen present in the natural atmosphere with varying concentrations of oxygen, carbon dioxide, or numerous inert or other gases. From a regulatory standpoint, the establishment would hopefully be overseen and classified as equivalent to recreational diving.

1. The commodity of a business establishment or residence comprised of a hyperbaric breathable atmosphere.

2. The result of claim 1 wherein the intention is to provide an alcohol-like experience without the use of alcohol.

3. The result of claim 1 while adjusting the natural atmospheric concentrations of oxygen and nitrogen with various alternate gases such as carbon dioxide, xenon, and others.

4. The result of claim 1 whereby different areas within the establishment can maintain different assortments of partial pressures of oxygen and various gases.

5. The result of claim 1 whereby the intoxicating effect is readily adjustable.

6. The result of claim 1 whereby the location of the establishment is located underground.

7. The result of claim 1 for the purpose of providing residence for people suffering from Chronic Obstructive Pulmonary Disease.

8. The purchase of subterranean real estate as distinct from surface real estate.
